(19) United States
(12) Patent Application Publication
(10) Pub. No.: US 2011/0206623 A1
(43) Pub. Date: Aug. 25, 2011

(54) NOVEL DIPOLAR IONIC COMPOUNDS COMPRISING FORMULATIONS AND THE USE THEREOF

(75) Inventors: Hans Henning Wenk, Mulheim an der Ruhr (DE); Holger Leidreiter, Hattingen (DE); Mike Farwick, Essen (DE); Petra Allef, Krefeld (DE); Ursula Maczkiewitz, Essen (DE); Sascha Herrwerth, Essen (DE)

(73) Assignee: EVONIK GOLDSCHMIDT GMBH, Essen (DE)

(21) Appl. No.: 12/674,831

(22) PCT Filed: Jun. 24, 2008
(86) PCT No.: PCT/EP08/58003

§ 371 Date: May 9, 2011

(30) Foreign Application Priority Data
Aug. 23, 2007 (DE) 102007040001.4

(51) Int. Cl. A61K 8/44; C07C 229/26; A61Q 17/04; A61Q 19/10; A61Q 19/00

(52) U.S. Cl. 424/59; 562/561; 514/556; 510/130

(57) ABSTRACT

A subject-matter of the invention is formulations comprising novel zwitterionic compounds and the use of these formulations as cosmetics.

### FIELD OF THE INVENTION

A subject-matter of the invention is formulations comprising novel zwitterionic compounds and also the use of these formulations as cosmetics.

### STATE OF THE ART

The surface-active glycinate compounds known to date, such as, e.g., cocamidopropyl betaines, are used, for example, as amphoteric surfactants, in particular for hair and skin cleaning preparations, such as shampoos skin-friendly foam and shower gels, and personal and body care products.

Inter alia, these improve the dermatological properties of anionic and nonionic surfactants and give the skin a pleasant feeling.

In addition, betaines can advantageously be used in cleaning products, such as dishwashing formulations and mild detergents.

Use may in particular be made, as betaines of the state the art, of fatty acid amidopropyl betaines, the fatty acid residues of which exhibit, in the mixture, generally from 8 to 18 carbon atoms. Compounds of this type are described, for example, in EP 711 545.

Because of their surface-active properties, betaines according to the state of the art have the capability of forming a thick and creamy foam, which remains stable for a long period of time even in the presence of other surfactants, soaps and additives, combined with good cleaning properties without irritant side effects.

The preparation of betaines is described in detail in the relevant patent and specialist literature (U.S. Pat. No. 3,225,074). Generally, in this connection, compounds comprising tertiary amine nitrogen atoms are reacted with co-halocarboxylic acids or the salts thereof in aqueous or water-comprising media.

Use is made in particular, as compounds comprising tertiary amine nitrogen atoms, of fatty acid amides of the general formula

R³—CONH—(CH₂)ₘ—NR⁴R⁵

in which R³ is the alkyl radical of a fatty acid, R⁴ and R⁵ are identical or different alkyl radicals with 1-4 carbon atoms and m can be 1-3.

In this connection, the alkyl radical R³ is usually derived from natural or synthetic fatty acids with 6-20 carbon atoms and the mixtures thereof.

Suitable fatty acids are, for example, caprylic acid, capric acid, lauric acid, palmitic acid, stearic acid, behenic acid, linoleic acid, caproic acid, linolenic acid or ricinoleic acid.

The naturally occurring fatty acid mixtures with a chain length of 8-18 carbon atoms, such as coconut oil fatty acid or palm kernel oil fatty acid, which, if appropriate, can be hardened by suitable hydrogenation methods, often have a use.

The horny layer (stratum corneum, SC), which represents the outermost layer of the skin, is, as important barrier layer, of particular importance in protecting from environmental influences. The skin requires an optimum of water in order to maintain its smoothness, elasticity and suppleness. These findings were confirmed in fundamental work inter glia by Jacobi and also Schuleit and Szakall (Jacobi, J. Appl. Physiol., 12 (3), 403-7, May 1958; Schneider W & Schuleit H, Arch. Klein. Exp. Dermatol., 193 (5), 434-59, December 1951; Szakall A, Arch. Klein. Exp. Dermatol., 206, 374-9, 1957).

Every day, a human being loses from several decilitres to several litres of water to the outside world through the skin. The water present in the skin originates from various sources and, according to relatively recent findings, is present both as vapour and in liquid form, and also adsorbed on proteins. It is not known how much water the epidermis comprises; however, it can be assumed that a water content of up to 30% is present in some layers of the stratum corneum.

It can be accepted as certain that water is capable of migrating through different layers of the skin. In this connection, various models exist for the diffusion of water through the layers of the skin, not one of which to date has been able to be conclusively proven:

Analogously to hydrophobic substances, which can penetrate into the horny layer through lipid pores, water is supposed to be transported through specific “aqueous pores”. These pores are supposed to have a diameter of 15-25 Å.

Another approach postulates that water-filled channels are supposed to pass through the stratum corneum. It has been possible to show, by X-ray diffraction experiments, that holes exist in a lipid bilayer system which are big enough for condensed water to be able to accumulate there.

Thus, in addition to an intact permeability barrier, the presence of water-binding substances which are formed in the epidermal horny layers is undoubtedly crucially necessary for the moisture regulation of the skin. These natural moisturizing factors. (NMF) present in the epidermis bind moisture in the skin. They represent a mixture of different compounds and consist of 40% amino acids, 12% pyrrolidonecarboxylic acid, 7% urea and 41% inorganic and organic salts, generally lactates.

Drastic environmental conditions, such as, e.g., low temperatures or too little humidity in winter, contribute to a considerable degree to the skin becoming raw and dry. Moreover, the moisturizing factors present in the epidermis are easily extracted by frequent washing or bathing. Thus, more water can escape from layers of the skin situated more deeply and the “transepidermal water loss” (TEWL) increases, resulting in desiccation of the skin. It is assumed that the loss of the natural moisturizing factors correlates with a reduction in the water content and a reduced softness of the keratin layer.

This is displayed sensorially by symptoms such as, e.g., a skin surface which appears increasingly raw, flaky, lacklustre and dull. A loss of flexibility and a harmful effect on the barrier function of the skin, which depends on the water binding capacity of the stratum corneum, are the result. The water content of the horny layer is thereby further reduced.

Scrupulous care in preventing the skin from being continually dry is not only an aesthetic requirement but also a proven means of effectively preventing chronic skin diseases. In this connection, the moisture regulating of the skin can be effectively assisted by topical application of appropriate formulations.

A multitude of in vivo methods are known for determining the moisture content of the skin. In this connection, physical parameters, such as the conductivity and the dielectric properties (capacity) of the horny layer, which directly correlate with the skin moisture are determined. Various measuring instruments are available for determining the hydration of the stratum corneum, such as, e.g., the corneometer types CM 820 and CM 825 (Courage+Khazaka) and also the Skicon 200 dermal phase meter (Nova). These noninvasive and simple methods allow a change in the skin moisture to be quantitatively measured. In addition, the elasticity of the skin can be determined via the Dermal Torque Meter (DiaStron) or also via the Cutometer (Courage+Khazaka).

There are a number of cosmetic formulations with a water-regulating effect for counteracting a dry state of the skin and restoring the water balance of the skin. These preparations are, in the form of emulsions, ideal formulations for supplying the skin with fat and moisture, and generally comprise a number of active substances which exhibit a protective function on application, thereby improving the condition of the skin surface and changing the functional condition of the skin by, e.g., having a regulating influence on the skin moisture and bringing about caring properties by penetration under the skin surface.

Various mechanisms exist for cosmetic ingredients and formulations to have a positive influence on the epidermal water content:

The evaporation of water from the upper layers of the skin can be prevented through an occlusive lipid or polymer film. Water is thereby provided to the upper layers of the skin by the lower layers of the skin and the formation of sweat is reduced, whereby the skin moisture of the upper layers of the SC is greatly increased. Under such occlusive conditions, however, a build-up of water in the skin and an increased endogenous swelling of the horny layer typically occur, whereby the ability of the skin to regenerate is slowed down.

With caring cosmetic formulations, it is possible in formulation terms to prepare cosmetic products which comprise more water than the stratum corneum and accordingly provide the SC with water on penetration of the intact formulation. Special lipids are likewise in a position to reduce transepidermal water loss and can accordingly also be regarded as a type of moisturizer.

A further conventional approach is the addition to cosmetic emulsions, gels or cleaning body care products of moisture-maintaining products as activating ingredients which should guarantee that the keratin layer is cared for with a sufficient moisture over defined periods of time. Moisture-maintaining products are also described as moisturizers or humectants and should, on the one hand, retain water in the epidermis and, on the other hand, reduce TEWL by stabilizing the barrier function in the upper horny layer.

A multitude of such substances are described and are already used. These generally have the ability to bind water more or less strongly and to completely or partially replace the natural substances which have been washed out. In principle, these include hygroscopic substances, such as, above all, polyhydric alcohols, ethoxylated polyols, sugars and also polysaccharides, such as, e.g., the endogenous moisture-maintaining product hyaluronic acid and its salts, which play an important role in moisture regulation since they can bind water in the stratum corneum. This results finally in an improvement in the skin elasticity.

In particular, body cleaning products, such as shower gels or shampoos, result in a major change in the lipid composition of the skin, resulting in a deterioration in the barrier function of the skin and accordingly in an increased transepidermal water loss. The literature describes a multitude of moisturizers which are used to compensate for this effect, such as, for example, Bis-PEG/PPG-20/20 Dimethicone (Abil® B 8832, Goldschmidt GmbH), glycerol or PEG-7 glyceryl cocoate (Tegosoft® GC, Goldschmidt GmbH).

On cleaning the skin, endogenous lipids are also washed out by the surfactants used, in addition to the lipophilic contamination. This effect is often felt as unpleasant; the skin feels raw and rough. The skin is also described as “dry”, the absence of fat, however, being meant here. Accordingly, “refatting agents” can be added to formulations according to the invention, especially body cleansing formulations with the result that the defatting process described is reduced. As a result, on the one hand, the fat washed out can be replaced by the refatting agents and, on the other hand, however, the defatting action of the formulation per se can also be reduced by the use of the refatter.

In formulation terms, it is difficult and accordingly unconventional to use cosmetic oils, such as, e.g., Tegosoft M® (Goldschmidt GmbH, isopropyl myristate), for this purpose as these oils have to be dissolved, which is expensive. Accordingly, use is preferably made, as conventional refatters, of more hydrophilic products, such as, e.g., Tegosoft GC® (Goldschmidt GmbH, PEG-7 glyceryl cocoate), which are already dissolved due to the excess of the cleaning surfactants. The analysis of a product database which includes worldwide product innovations in consumer markets (“Global New Products Database”: Mintel) revealed that 29% of all skin cleaning formulations on the European market (9/05-9/06) comprised PEG-7 glyceryl cocoate.

It is assumed that the refatting process takes place on rinsing off the formulation after the actual washing. In the process of rinsing with water, the existing solution is diluted until the CMC (critical micelle concentration) is fallen short of.

With the release of the micelle components (the lipophilic refatters, the surfactants and solubilizators), the refatters again become insoluble. These lipophilic substances (both endogenous lipids and emollients/cosmetic oils) precipitate and absorb on the skin.

Generally, an ideal moisturizer should already in a low concentration of use give rise to a marked effect, should be non-toxic and very well tolerated by the skin, should exhibit high compatibility with other ingredients, should exhibit good long term stability and should be able to be incorporated without problems in skin treatment products.

It is particularly desirable for a moisturizer to be able to be manufactured simply and economically; during production, it should be obtained in a form which is guaranteed to be simple to handle and in addition meets the high purity requirements placed on cosmetic or dermatological active substances. A moisturizer should exhibit additional multifunctional properties; thus, in addition to returning the water content of the skin to normal, it should in addition also exhibit, for example, protective, soothing or anti-inflammatory properties.

In spite of many years of research in the field of skin moisture-maintaining products, the substances used at present as moisture-maintaining products on close examination do not completely meet the demands placed on them.

### SUMMARY OF THE INVENTION

It is an object of the invention to make available novel moisturizers which meet the above criteria. In one embodiment, the present invention provides a formulation that includes at least one compound of formula I

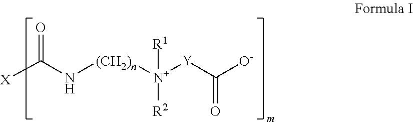

in which

n=1 to 6 and

m=1 to 4 and

R^{l} and R² are, independently of one another, identical or different aliphatic hydrocarbon radicals having 1 to 6 carbon atoms and

Y is a divalent hydrocarbon radical and

X is an m-valent radical or a covalent bond, with
X, for m=1, being a hydrogen or a C₁-C₄-hydrocarbon which is unsubstituted or substituted with at least one OH group and
X, for m=2, being a direct covalent bond or a divalent C₁-C₅-hydrocarbon radical which is unsubstituted or substituted with at least one OH group, and
X, for m>2, being an m-valent C₁-C₅-hydrocarbon radical which is unsubstituted or substituted with at least one OH group,

and/or a stereoisomeric form of the compound according to formula I.

### BRIEF DESCRIPTION OF THE DRAWINGS

FIG. 1 is a graph illustrating the LDH release after application of the test formulation within the examples of the present invention.

FIG. 2 is a graph illustrating the total LDH after 24 and 48 h after damaging with SDS.

FIG. 3 is a graph illustrating the IL-1α concentration 24 h after damaging.

FIG. 4 is a graph illustrating the sum of the IL-1α concentration 24 and 48 h after damaging with SDS.

FIG. 5 is a graph illustrating the viability of the cells 24 h after application of the test formulations twice.

FIG. 6 is a graph illustrating the viability of the skin cells 24 h after damaging with SDS.

FIG. 7 is a graph illustrating the water retention capacity of different short-chain zwitterionic compounds.

FIG. 8 is a graph illustrating the improvement in the skin moisture by compound 2.1.

FIG. 9 is a graph illustrating the long-term moisturizer effects of compound 2.1.

FIG. 10 is a graph illustrating the decrease in the protein concentration, based on the vehicle.

FIG. 11 is a graph illustrating the corneometry data of the panel test.

### DESCRIPTION OF THE INVENTION

It has been found, surprisingly, that the formulations described subsequently, which comprise short-chain zwitterionic compounds, result in an improvement in the condition of the skin and in particular in an improvement in the skin moisture. Particularly surprisingly, formulations according to the invention have an anti-inflammatory effect on damaged cells.

As stated above and in one embodiment, the present invention relates to formulations that include at least on compound of formula I and the use of such formulations as cosmetics.

In another embodiment, the present invention relates to the use of compounds according to formula I for increasing and/or stabilizing the moisture content of the skin.

The formulations according to the invention and the use thereof are described subsequently by way of example, without the invention being limited to these exemplary embodiments. If ranges, general formulae or categories of compounds are given subsequently, these are to comprise not only the corresponding ranges or groups of compounds which are explicitly mentioned but also all subranges and subgroups of compounds which can be obtained by extraction of individual values (ranges) or compounds. If documents are mentioned in the context of the present description, the content thereof is to be completely incorporated in the disclosure content of the present invention. “Short-chain” zwitterionic compounds are to be understood subsequently as those which exhibit, according to formula I, an X with≦5 carbon atoms. “Relatively long or long-chain” zwitterionic compounds are to be understood as those which exhibit an X with>5 carbon atoms. All percentages (%) given are percentages by weight, unless otherwise indicated.

Formulations according to the invention are characterized in that they comprise at least one compound according to formula I:

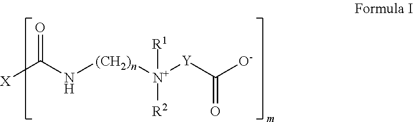

in which

n=1 to 6, preferably 1 to 3, preferably 3, and m=to 4, preferably 1 or 2, and R¹ and R² are,

independently of one another, identical or different aliphatic hydrocarbon radicals having 1 to 6 carbon atoms, preferably C₂-C₃-hydrocarbon radicals and preferably CH₃ radicals, and Y is a divalent hydrocarbon radical, preferably —CH₂—, and X is an m-valent radical or a covalent bond,

with: for m=1, X being a hydrogen or a C₁-C₄-hydrocarbon radical which is unsubstituted or substituted with at least one OH group, and also, for m=2, X being a direct bond, —CH₂—, —CH(OH)—, —CH₂CH(OH)— or —CH(OH)CH(OH)—, and X, for m=2, being a direct connection or a divalent C₁-C₅-hydrocarbon radical which is unsubstituted or substituted with at least one OH group, and X, for m>2, being an m-valent C₁-C₅-hydrocarbon radical which is unsubstituted or substituted with at least one OH group, and/or a stereoisomeric form of the compound according to formula I.

If formulations according to the invention comprise at least one compound of the formula I in which m=2, X is preferably a direct covalent bond, CH₂, CH(OH), CH₂CH(OH) or CH(OH)CH(OH), preferably CH₂.

If formulations according to the invention comprise at least one compound of the formula I in which m=1, X is preferably ethyl, hydroxymethyl, 1-hydroxyethyl, 2-hydroxyethyl, 1-hydroxypropyl, 2-hydroxypropyl or 3-hydroxypropyl and particularly preferably H.

Formulations according to the invention preferably comprise compounds according to formula I in which n=3.

Particularly preferred formulations according to the invention are those comprising at least one compound according to formula I in which R¹=R²═CH₃.

Formulations according to the invention preferably comprise at least one compound according to formula I in which Y=CH₂.

Particular preference is given to formulations comprising at least one compound according to formula I in which n=3, m=2, R¹=R²=CH₃, Y=CH₂ and X=CH₂ or n=3, m=1, R¹=R²=CH₃, Y=CH₂ and X=H.

Formulations according to the invention preferably comprise at least one compound of the formula I in an amount of 0.05 to 10% by weight and preferably in an amount of 0.1 to 5% by weight, based on the total formulation.

Formulations according to the invention can, e.g., comprise at least one additional component chosen from the group consisting of
emollients
emulsifiers and surfactants
thickeners/viscosity regulators/stabilizers
UV/light screening agents
antioxidants
hydrotropes (or polyols)
solids
pearlescent additives
deodorant and antiperspirant active substances
insect repellents
self-tanning agents
preservatives
conditioners
fragrances
colorants
biogenic active substances
care additives
solvents.

Use may be made, as emollients, of all cosmetic oils, in particular mono- or diesters of linear and/or branched mono- and/or dicarboxylic acids having 2 to 44 carbon atoms with saturated or unsaturated and linear and/or branched alcohols having 1 to 22 carbon atoms. Use may likewise be made of the esterification products of bifunctional aliphatic alcohols having 2 to 36 carbon atoms with monofunctional aliphatic carboxylic acids having 1 to 22 carbon atoms. Furthermore, long-chain arylcarboxylic acid esters, such as, e.g., esters of benzoic acid, e.g. benzoic acid esters of saturated or unsaturated and linear or branched alcohols having 1 to 22 carbon atoms, or also isostearyl benzoate or octyldodecyl benzoate, are suitable. Additional monoesters suitable as emollients and oil components are, e.g., the methyl esters and isopropyl esters of fatty acids having 12 to 22 carbon atoms, such as, e.g., methyl laurate, methyl stearate, methyl oleate, methyl erucate, isopropyl palmitate, isopropyl myristate, isopropyl stearate or isopropyl oleate. Other suitable monoesters are, e.g., n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate or erucyl oleate, and also esters which can be obtained from industrial aliphatic alcohol cuts and industrial aliphatic carboxylic acid mixtures, e.g. esters of unsaturated fatty alcohols having 12 to 22 carbon atoms and saturated and unsaturated fatty acids having 12 to 22 carbon atoms, such as are accessible from animal and vegetable fats. However, naturally occurring monoester or wax ester mixtures, such as are present, e.g., in jojoba oil or in sperm oil, are also suitable. Suitable dicarboxylic acid esters are, e.g., di(n-butyl) adipate, di(n-butyl) sebacate, di(2-ethylhexyl) adipate, di(2-hexyldecyl) succinate or diisotridecyl azelate. Suitable diol esters are, e.g., ethylene glycol dioleate, ethylene glycol diisotri-decanoate, propylene glycol di(2-ethylhexanoate), butanediol diisostearate and neopentyl glycol dicaprylate.

Additional fatty acid esters which can be used as emollients are, e.g., C₁₂₋₁₅-alkyl benzoate, dicaprylyl carbonate or diethylhexyl carbonate. Use may likewise be made, as emollients and oil components, of relatively long-chain triglycerides, i.e. triple esters of glycerol with three acid molecules, at least one of which is a relatively long-chain acid molecule. Mention may be made here, by way of example, of fatty acid triglycerides; use may be made as such, as emollients and oil components, of, for example, natural vegetable oils, e.g. olive oil, sunflower oil, soybean oil, peanut oil, rapeseed oil, almond oil or palm oil, but also the liquid portion of coconut oil or palm kernel oil, and also animal oils, such as, e.g., neatsfoot oil or the liquid portions of beef tallow, or also synthetic triglycerides of caprylic/capric acid mixtures, triglycerides of industrial oleic acid, triglycerides with isostearic acid or triglycerides of palmitic/oleic acid mixtures. Use may furthermore be made of hydrocarbons, in particular also liquid paraffins and isoparaffins. Examples of hydrocarbons which can be used are paraffin oil, isohexadecane, polydecene, petroleum jelly, light liquid paraffin or squalane. Use may further also be made of linear or branched fatty alcohols, such as oleyl alcohol or octyldodecanol, and also fatty alcohol ethers, such as dicaprylyl ether. Suitable silicone oils and waxes are, e.g., polydimethylsiloxanes, cyclomethylsiloxanes and also aryl- or alkyl- or alkoxy-substituted polymethyl-siloxanes or cyclomethylsiloxanes.

Use may be made, as emulsifiers or surfactants, of nonionic, anionic, cationic or amphoteric surfactants.

Use may be made, as nonionic emulsifiers or surfactants, of compounds from at least one of the following groups:
Addition products of 2 to 100 mol of ethylene oxide and/or 0 to 5 mol of propylene oxide with linear fatty alcohols having 8 to 22 carbon atoms, with fatty acids having 12 to 22 carbon atoms and with alkylphenols having 8 to 15 carbon atoms in the alkyl group
C_{12/19}-Fatty acid mono- and diesters of addition products of 1 to 100 mol of ethylene oxide with glycerol
Glycerol mono- and diesters and sorbitan mono- and diesters of saturated and unsaturated fatty acids having 6 to 22 carbon atoms and the ethylene oxide addition products thereof
Alkyl mono- and oligoglycosides having 8 to 22 carbon atoms in the alkyl radical and the ethylene oxide addition products thereof
Addition products of 2 to 200 mol of ethylene oxide with castor oil and/or hydrogenated castor oil
Partial esters based on unsaturated or saturated and linear or branched C₆-C₂₂-fatty acids, ricinoleic acid and also 12-hydroxystearic acid and glycerol, polyglycerol, pentaerythritol, dipentaerythritol, sugar alcohols (e.g. sorbitol), alkyl glucosides (e.g. methyl glucoside, butyl glucoside or lauryl glucoside) and polyglucosides (e.g. cellulose)
Mono-, di- and trialkyl phosphates and also mono-, di- and/or tri(PEG-alkyl) phosphates and the salts thereof
Polysiloxane/polyether copolymers (dimethicone copolyols), such as, e.g., PEG/PPG-20/6 Dimethicone, PEG/PPG-20/20 Dimethicone, Bis-PEG/PPG-20/20 Dimethicone, PEG-12 or PEG-14 Dimethicone, PEG/PPG-14/4 or 4/12 or 20/20 or 18/18 or 17/18 or 15/15 dimethicone
Polysiloxane/polyalkyl/polyether copolymers or corresponding derivatives, such as, e.g., lauryl or cetyl dimethicone copolyols, in particular cetyl PEG/PPG-10/1 dimethicone (Abil® EM 90 (Degussa))
Mixed esters of pentaerythritol, fatty acids, citric acid and fatty alcohol according to DE-PS 11 65 574 and/or mixed esters of fatty acids having 6 to 22 carbon atoms, methyl glucose and polyols, preferably glycerol or polyglycerol
Citric acid esters, such as, e.g., glyceryl stearate citrate, glyceryl oleate citrate and dilauryl citrate.

Anionic emulsifiers or surfactants can comprise water-solubilizing anionic groups, such as, e.g., a carboxylate, sulphate, sulphonate or phosphate group, and a lipophilic radical. A large number of anionic surfactants compatible with the skin are known to a person skilled in the art and are available commercially. In this connection, they can be alkyl sulphates or alkyl phosphates in the form of their alkali metal, ammonium or alkanolammonium salts, alkyl ether sulphates, alkyl ether carboxylates, acyl-sarcosinates and also sulphosuccinates and acyl-glutamates in the form of their alkali metal or ammonium salts.

Cationic emulsifiers and surfactants can also be added. In particular, use may be made, as such, of quaternary ammonium compounds, in particular those provided with at least one saturated or unsaturated and linear and/or branched alkyl chain having 8 to 22 carbon atoms; thus, for example, alkyltrimethylammonium halides, such as, e.g., cetyltrimethylammonium chloride or bromide or behenyltrimethylammonium chloride, but also dialkyl-dimethylammonium halides, such as, e.g., distearyl-dimethylammonium chloride, can be used. In addition, monoalkylamidoquats, such as, e.g., palmitamidopropyltrimethylammonium chloride, or corresponding dialkylamidoquats can be used. In addition, use may be made of quaternary ester compounds which biodegrade well and which can be quaternized fatty acid esters based on mono-, di- or triethanolamine. Furthermore, alkylguanidinium salts can be installed as cationic emulsifiers.

It is furthermore possible to use amphoteric surfactants, such as, e.g., betaines, amphoacetates or amphopropionates, together with the polyglycerol esters according to the invention.

All thickening agents known to a person skilled in the art are possible as thickeners for the thickening of oil phases. Mention may in particular be made, in this connection, of waxes, such as hydrogenated castor wax, beeswax or microcrystalline wax. Furthermore, use may also be made of inorganic thickening agents, such as silica, alumina or layered silicates (e.g. hectorite, laponite or saponite). These inorganic oil-phase thickeners can in this connection by hydrophobically modified. Use may in this connection be made, for the thickening/stabilizing of water-in-oil emulsions, of in particular aerosils, layered silicates and/or metal salts of fatty acids, such as, e.g., zinc stearate.

Possible viscosity regulators for aqueous surfactant systems include, e.g., NaCl, low molecular weight nonionic surfactants, such as cocamide DEA/MEA and laureth-3, or polymeric high molecular weight associative highly-ethoxylated fatty derivatives, such as PEG-200 hydrogenated glyceryl palmate.

Use may be made, as UV/light screening agents, for example, of organic substances which are in a position to absorb ultraviolet radiation and to readmit the absorbed energy in the form of longer wavelength radiation, e.g. heat. UV-B screening agents may be oil-soluble or water-soluble. Mention may be made, as oil-soluble UV-B/light screening agents, e.g., of:
3-Benzylidenecamphor and the derivatives thereof, e.g. 3-(4-methylbenzylidene)camphor
4-Aminobenzoic acid derivatives, such as, e.g., 2-ethylhexyl 4-(dimethylamino)benzoate and amyl 4-(dimethylamino)benzoate
Cinnamic acid esters, such as, e.g., 2-ethylhexyl 4-methoxycinnamate, isopentyl 4-methoxycinnamate or 2-ethylhexyl 2-cyano-2-phenylcinnamate (octocrylene)
Salicylic acid esters, such as, e.g., 2-ethylhexyl salicylate, 4-isopropylbenzyl salicylate or homo-menthyl salicylate
Benzophenone derivatives, such as, e.g., 2-hydroxy-4-methoxybenzophenone, 2-hydroxy-4-methoxy-4′-methylbenzophenone or 2,2′-dihydroxy-4-methoxybenzophenone
Benzalmalonic acid esters, such as, e.g., di(2-ethylhexyl) 4-methoxybenzalmalonate
Triazine derivatives, such as, e.g., 2,4,6-trianilino-(p-carbo-2′-ethyl-1′-hexyloxy)-1,3,5-triazine and octyl triazone
Propane-1,3-diones, such as, e.g., 1-(4-(tert-butyl)phenyl)-3-(4′-methoxyphenyl)propane-1,3-dione.

Possible water-soluble UV-B/light screening agents are:
2-Phenylbenzimidazole-5-sulphonic acid and the alkali metal, alkaline earth metal, ammonium, alkylammonium, alkanolammonium and glucammonium salts thereof
Sulphonic acid derivatives of benzophenone, such as, e.g., 2-hydroxy-4-methoxybenzophenone-5-sulphonic acid and its salts
Sulphonic acid derivatives of 3-benzylidenecamphor, such as, e.g., 4-(2-oxo-3-bornylidenemethyl)benzenesulphonic acid and 2-methyl-5-(2-oxo-3-bornylidene)benzenesulphonic acid and the salts thereof.

Derivatives of benzoylmethane are possible in particular as typical UV-A/light screening agents, such as, for example, 1-(4′-(tert-butyl)phenyl)-3-(4′-methoxyphenyl)propane-1,3-dione or 1-phenyl-3-(4′-isopropylphenyl)propane-1,3-dione. The UV-A and UV-B screening agents can obviously also be used in mixtures.

In addition to the soluble substances mentioned, insoluble pigments, namely finely dispersed metal oxides or salts, are also possible for this purpose, such as, for example, titanium dioxide, zinc oxide, iron oxide, aluminium oxide, cerium oxide, zirconium oxide, silicates (talc), barium sulphate and zinc stearate. The particles should, in this connection, exhibit a mean diameter of less than 100 nm, e.g. between 5 and 50 nm and in particular between 15 and 30 nm. They may exhibit a spherical form; however, use may also be made of those particles which have an ellipsoidal form or a form deviating in another way from the spherical shape. A relatively new category of light screening agents comprises micronized organic pigments, such as, for example, 2,2′-methylenebis{6-(2H-benzotriazol-2-yl)-4-(1,1,3,3-tetramethylbutyl)phenol}, with a particle size of less than 200 nm, which is available, e.g., as a 50% aqueous dispersion.

Additional suitable UV/light screening agents can be found in the review by P. Finkel in SOFW-Journal 122, 543 (1996).

In addition to the two groups of primary UV/light screening agents mentioned above, secondary light screening agents of the antioxidants type can also be used which interrupt the photochemical reaction chain, which is triggered if UV radiation penetrates the skin. Use may be made, as antioxidants, e.g., of superoxide dismutase, tocopherols (vitamin E), dibutylhydroxytoluene and ascorbic acid (vitamin C).

Use may be made, as hydrotropes for improving the flow behaviour and the application properties, for example of ethanol, isopropyl alcohol or polyols. Polyols which are suitable here can have from 2 to 15 carbon atoms and at least two hydroxyl groups. Typical examples are:
Glycerol alkylene glycols, such as, for example, ethylene glycol, diethylene glycol, propylene glycol, butylene glycol, hexylene glycol and also polyethylene glycols with an average molecular weight of 100 to 1000 daltons
Industrial oligoglycerol mixtures with a degree of self-condensation of 1.5 to 10, such as, for example, industrial diglycerol mixtures with a diglycerol content of 40 to 50% by weight
Methylol compounds, such as, in particular, trimethylolethane, trimethylolpropane, trimethylolbutane, pentaerythritol and dipentaerythritol
Low alkyl glucosides, in particular those having 1 to 4 carbon atoms in the alkyl radical, such as, for example, methyl and butyl glucoside
Sugar alcohols having 5 to 12 carbon atoms, such as, for example, sorbitol or mannitol
Sugar having 5 to 12 carbon atoms, such as, for example, glucose or sucrose
Amino sugars, such as, for example, glutamine.

Use may be made, as solids, for example of iron oxide pigments, titanium dioxide or zinc oxide particles and those additionally mentioned under “UV protecting agents”. Furthermore, use may also be made of particles which result in special sensory effects, such as, for example, nylon-12, boron nitride, polymer particles, such as, for example, polyacrylate or polymethacrylate particles, or silicone elastomers.

Use may be made, as pearlescent additives, e.g., of glycol distearates or PEG-3 distearate.

Possible deodorant active substances include, e.g., odour-masking agents, such as the common constituents of fragrances, odour absorbers, for example the layered silicates described in the laid-open patent specification DE-P 40 09 347, in particular among these montmorillonite, kaolinite, ilite, beidelite, nontronite, saponite, hectorite, bentonite or smectite, and furthermore, for example, zinc salts of ricinoleic acid. Germicidal agents are likewise suitable for incorporation. Germicidal substances are, for example, 2,4,4′-trichloro-2′-hydroxydiphenyl ether (irgasan), 1,6-di(4-chlorophenylbiguanido)hexane (chlorhexidin), 3,4,4′-trichlorocarbanilide, quaternary ammonium compounds, clove oil, peppermint oil, thyme oil, triethyl citrate, farnesol (3,7,11-trimethyl-2,6,10-dodecatrien-1-ol), ethylhexyl glyceryl ether, polyglyceryl-3 caprylate (Tego® Cosmo P813, Degussa), and also the active agents described in the laid-open patent specifications DE 198 55 934, DE-37 40 186, DE-39 38 140, DE-42 04 321, DE-42 29 707, DE-42 29 737, DE-42 38 081, DE-43 09 372, DE-43 24 219 and EP 666 732.

Use may be made, as antiperspirant active substances, of astringents, for example basic aluminium chlorides, such as aluminium chlorohydrate (“ACH”) and aluminium/zirconium/glycine salts (“AZG”).

Use may be made, as insect repellents, for example of N,N-diethyl-m-toluamide, 1,2-pentanediol or insect repellent 3535.

Use may be made, as self-tanning agents, e.g. of dihydroxyacetone and erythrulose.

Use may be made, as preservatives, for example of mixtures of individual or several alkylparaben esters with phenoxyethanol. The alkylparaben esters can be methylparaben, ethylparaben, propylparaben and/or butylparaben. Use may also be made, in place of phenoxyethanol, of other alcohols, such as, for example, benzyl alcohol or ethanol. In addition, use may also be made of other normal preserving agents, such as, for example, sorbic or benzoic acid, salicylic acid, 2-bromo-2-nitropropane-1,3-diol, chloroacetamide, diazolidinyl urea, DMDM hydantoin, iodopropynyl butylcarbamate, sodium hydroxymethylglycinate, methylisothiazoline, chloromethylisothiazoline, ethylhexylglycerine or caprylyl glycol.

Use may be made, as conditioning agents, e.g., of organic quaternary compounds, such as cetrimonium chloride, dicetyldimonium chloride, behentrimonium chloride, distearyldimonium chloride, behentrimonium methosulfate, distearoylethyldimonium chloride, palmitamidopropyltrimonium chloride, guar hydroxypropyltrimonium chloride, hydroxypropyl guar hydroxypropyltrimonium chloride or quaternium-80, or also amine derivatives, such as, e.g., aminopropyl dimethicone or stearamidopropyl dimethylamines.

Use may be made, as fragrances, of natural or synthetic odoriferous substances or mixtures thereof. Natural odoriferous substances are extracts of flowers (lily, lavender, rose, jasmine, neroli or ylang-ylang), stems and leaves (geranium, patchouli, petitgrain), fruits (anis, coriander, caraway, juniper), fruit rinds (bergamot, lemon, orange), roots (mace, angelica, celery, cardamom, costus, iris, thyme), needles and twigs (spruce, fir, pine, mountain pine) and resins and balsams (galbanum, elemi, benzoin, myrrh, frankincense, opoponax). Animal raw materials are also possible, such as, for example, civet and castoreum. Typical synthetic perfume compounds are products of the ester, ether, aldehyde, ketone, alcohol and hydrocarbon types. Perfume compounds of the ester type are, e.g., benzyl acetate, phenoxyethyl isobutyrate, p-(tert-butyl)cyclohexyl acetate, linalyl acetate, dimethylbenzylcarbinyl acetate, phenylethyl acetate, linalyl benzoate, benzyl formate, ethylmethyl phenylglycinate, allylcyclohexyl propionate, styrallyl propionate and benzyl salicylate. The ethers include, for example, benzyl ethyl ether, the aldehydes include, e.g., linear alkanals having 8 to 18 carbon atoms, citral, citronellal, citronellyloxyacetaldehyde, cyclamen aldehyde, hydroxycitronellal, lilial and bourgeonal, the ketones include, e.g., the ionones, α-isomethyl ionone and methyl cedryl ketone, the alcohols include anethole, citronellol, eugenol, isoeugenol, geraniol, linalool, phenylethyl alcohol and terpineol, and the hydrocarbons include mainly the terpenes and balsams. Use may be made of mixtures of different odoriferous substances which together generate an attractive scent. Essential oils of low volatility, which are generally used as flavouring components, are also suitable as fragrances, e.g. sage oil, camomile oil, clove oil, balm oil, peppermint oil, cinnamon leaf oil, linden blossom oil, juniper berry oil, vetiver oil, frankincense oil, galbanum oil, labdanum oil and lavandin oil. Use may be made of bergamot oil, dihydromyrcenol, lilial, lyral, citronellol, phenylethyl alcohol, α-hexylcinnamaldehyde, geraniol, benzylacetone, cyclamen aldehyde, linalool, Boisambrene Forte, Ambroxan, indole, Hedione, Sandelice, lemon oil, mandarin oil, orange oil, allyl amyl glycolate, cyclovertal, lavandin oil, clary sage oil, β-damascone, geranium oil Bourbon, cyclohexyl salicylate, Vertofix Coeur, Iso-E-Super, Fixolide NP, Evernyl, Iraldein gamma, phenylacetic acid, geranyl acetate, benzyl acetate, rose oxide, Romillat, Irotyl and Floramat, alone or in mixtures.

Use may be made, as colorants, of the substances suitable and authorized for cosmetic purposes, such as are compiled, for example, in the publication “Kosmetische Färbemittel” [Cosmetic Colouring Agents] of the Farbstoffkommission der Deutschen Forschungsgemeinschaft [Colorant Commission of the German Research Association], Verlag Chemie, Weinheim, 1984, pp. 81-106. These colorants are only used in concentrations of 0.001 to 0.1% by weight, based on the complete mixture.

The term “biogenic active substances” is to be understood as meaning, for example, tocopherol and derivatives, ascorbic acid and derivatives, retinol and derivatives, deoxyribonucleic acid, coenzyme Q10, bisabolol, allantoin, phytantriol, panthenol, α-hydroxy acids, salicylic acid, amino acids, amino acid derivatives, hyaluronic acid, glucans, creatine and creatine derivatives, guanidine and guanidine derivatives, ceramides, phytosphingosine and phytosphingosine derivatives, sphingosine and sphingosine derivatives, pseudoceramides, essential oils, peptides, protein hydrolysates, plant extracts and vitamins and vitamin mixtures. These substances can be combined in any proportions with the novel zwitterionic compounds described.

It is possible to have present, as care additives, e.g., ethoxylated glycerol fatty acid esters, such as, for example, PEG-7 glycerol cocoate, or cationic polymers, such as, for example, polyquaternium-7, or polyglycerol esters.

Use may be made, as solvents, e.g., of propylene glycol, dipropylene glycol, glycerol, glycerol carbonate, water, ethanol, propanol or 1,3-propanediol.

A subject-matter of the invention is the use of the formulations according to the invention as cosmetics.

The compounds of the formula I can be present here preferably in a concentration of 0.05 to 10% by weight.

The formulation can be prepared as an emulsion; a typical emulsion (W/O or O/W) can, for example, comprise:
0.05 to 10% by weight of compounds of the formula I,
0 to 10% by weight, preferably >0 to 10% by weight, of one or more emulsifiers,
0 to 10% by weight, preferably >0 to 10% by weight, of one or more viscosity regulators or thickeners,
0 to 30% by weight, preferably >0 to 10% by weight, of one or more oily substances or emollients,
and also the usual auxiliaries and additives in the usual concentrations, and
ad 100% by weight of solvents.

Preferred emulsifiers and surfactants are the following nonionic, anionic, cationic or amphoteric surfactants:
Addition products of 2 to 100 mol of ethylene oxide and/or 0 to 5 mol of propylene oxide with linear fatty alcohols having 8 to 22 carbon atoms or with fatty acids having 12 to 22 carbon atoms,
C_{12/18}-fatty acid mono- and diesters of addition products of 1 to 100 mol of ethylene oxide with glycerol,
glycerol mono- and diesters and sorbitan mono- and diesters of saturated and unsaturated fatty acids having 6 to 22 carbon atoms and the ethylene oxide addition products thereof,
alkyl mono- and oligoglycosides having 8 to 22 carbon atoms in the alkyl radical and the ethylene oxide addition products thereof,
addition products of 2 to 200 mol of ethylene oxide with castor oil and/or hydrogenated castor oil,
partial esters based on unsaturated or saturated and linear or branched C₆-C₂₂-fatty acids, ricinoleic acid and also 12-hydroxystearic acid and glycerol, polyglycerol, pentaerythritol, dipentaerythritol, sugar alcohols (e.g. sorbitol), alkyl glucosides (e.g., methyl glucoside) and also polyglucosides (e.g. cellulose) and polyglyceryl-3 methylglucose distearate (Tego® Care 450 (Degussa)),
mono-, di- and trialkyl phosphates and also mono-, di- and/or tri(PEG-alkyl) phosphate and the salts thereof,
polysiloxane/polyether copolymers (dimethicone copolyols), bis-PEG/PPG-20/20 dimethicone, PEG-12 or PEG-14 dimethicone, PEG/PPG-14/4 or 4/12 or 20/20 or 18/18 or 17/18 or 15/15 dimethicone and bis-PEG/PPG-14/14 dimethicone or bis-PEG/PPG-16/16 and PEG/PPG-16/16 dimethicone,
polysiloxane/polyalkyl/polyether copolymers or corresponding derivatives, such as, e.g., lauryl or cetyl dimethicone copolyols, in particular cetyl PEG/PPG-10/1 dimethicone (Abil® EM 90 (Degussa)),
citric acid esters, such as, e.g., glyceryl stearate citrate, glyceryl oleate citrate and dilauryl citrate,
anionic emulsifiers or surfactants with water-solubilizing anionic groups, such as, e.g., a carboxylate, sulphate, sulphonate or phosphate group, and a lipophilic radical, such as, e.g., alkyl sulphates or alkyl phosphates in the form of their alkali metal, ammonium or alkanolammonium salts, alkyl ether sulphates, alkyl ether carboxylates, acylsarcosinates and also sulpho- and acylglutamates in the form of their alkali metal or ammonium salts,
cationic emulsifiers and surfactants, such as, e.g., quaternary ammonium compounds, for example alkyltrimethylammonium halides, such as, e.g., cetyltrimethylammonium chloride or behenyltrimethylammonium chloride, or dialkyldimethylammonium halides, such as, e.g., distearyldimethylammonium chloride,
monoalkylamidoquats, such as, e.g., palmitamidopropy trimethylammonium chloride, or corresponding dialkylamidoquats,
quaternary ester compounds which can biodegrade well, such as, e.g., quaternized fatty acid esters based on mono-, di- or triethanolamine, and
alkylguanidinium salts, amphoteric surfactants, such as, e.g., betaines, amphoacetates or amphopropionates.

Preferred emollients are:
mono- or diesters of linear and/or branched mono- and/or dicarboxylic acids having 2 to 44 carbon atoms with saturated or unsaturated and linear and/or branched alcohols having 1 to 22 carbon atoms,
esterification products of difunctional aliphatic alcohols having 2 to 36 carbon atoms with monofunctional aliphatic carboxylic acids having 1 to 22 carbon atoms,
methyl esters and isopropyl esters of fatty acids having 12 to 22 carbon atoms,
isopropyl palmitate, isopropyl myristate, isopropyl stearate, n-hexyl laurate, n-decyl oleate, isocetyl palmitate, isononyl palmitate, isononyl isononanoate, 2-ethylhexyl palmitate or oleyl erucate,
esters which can be obtained from industrial aliphatic alcohol cuts and industrial aliphatic carboxylic acid mixtures, e.g. esters of unsaturated fatty alcohols having 12 to 22 carbon atoms and saturated and unsaturated fatty acids having 12 to 22 carbon atoms, such as are extractable from animal and vegetable fats,
naturally occurring monoester or wax ester mixtures, such as are present, e.g., in jojoba oil or sperm oil,
dicarboxylic acid esters, such as, e.g., di(n-butyl) adipate, di(n-butyl) sebacate or di(2-ethylhexyl) adipate,
diol esters, such as, e.g., ethylene glycol dioleate,
propylene glycol di(2-ethylhexanoate), dicaprylyl carbonate, diethylhexyl carbonate or diisononyl carbonate,
triglycerides with isostearic acid; fatty acid triglycerides, such as, for example, natural vegetable oils, e.g. olive oil, sunflower oil, soybean oil, peanut oil, rapeseed oil, almond oil, palm oil or avocado oil; synthetic triglycerides of caprylic/capric acid mixtures,
liquid paraffins and isoparaffins,
isohexadecane, polydecene, petroleum jelly, light liquid paraffin or squalane,
linear or branched fatty alcohols, such as oleyl alcohol or octyldodecanol, and also fatty alcohol ethers, such as dicaprylyl ether,
silicone oils and waxes, such as, e.g., polydimethylsiloxanes, cyclomethylsiloxanes and also aryl- or alkyl- or alkoxy-substituted polymethylsiloxanes or cyclomethylsiloxanes,
di-PPG-3 myristyl ether adipate or PPG-3 benzyl ether myristate, propoxylated emollients, such as, e.g., PPG-3 myristyl ether, PPG-11 stearyl ether, PPG-15 stearyl ether or PPG-14 butyl ether.

Preferred viscosity regulators are:
NaCl, low molecular weight nonionic surfactants, such as cocamide DEA/MEA and laureth-3, or polymeric high molecular weight associative highly ethoxylated fatty derivatives, such as PEG-200 hydrogenated glyceryl palmate.

Preferred thickeners for the thickening of oil phases are:
waxes, such as hydrogenated castor wax, beeswax or microcrystalline wax,
inorganic thickening agents, such as silica, alumina or layered silicates, and hydrophobically modified ones,
aerosils, layered silicates and/or metal salts of fatty acids, such as, e.g., zinc stearate.

Formulations according to the invention can be hair care formulations, such as shampoos and/or conditioners, which exert a soothing action on irritable scalp skin.

The formulations according to the invention can also be used in cosmetic cleaning products.

Formulations according to the invention, in particular those for use as cosmetic cleaning product, such as, for example, shower gels, liquid soaps, face cleansers or bath shampoos, can comprise, for example:
0.05 to 10% by weight of compounds of the formula I,
3 to 20% by weight of one or more surfactants,
0 to 10% by weight, preferably >0 to 10% by weight, of one or more viscosity regulators,
0 to 10% by weight, preferably >0 to 10% by weight, of one or more conditioners for caring for the skin,
   and also the usual auxiliaries and additives in the usual concentrations, and
ad 100% by weight of solvents.

Preferred surfactants are anionic, amphoteric, nonionic and zwitterionic in structure. Preferred anionic surfactants can be the salts of different cations (sodium, ammonium or others) of alkyl sulphates or alkyl ether sulphates, such as lauryl sulphate, lauryl ether sulphate or myristyl ether sulphate, or sulphosuccinic acid derivatives. Preferred zwitterionic surfactants are, inter alia, cocamidopropyl betaine or sultaine. Preferred amphoteric surfactants are amphoacetates or glycinates, such as, e.g., sodium cocoamphoacetate or disodium cocoamphodiacetate.

Preferred nonionic surfactants can, for example, be alkyl polyglycosides, polyether derivatives (ethoxylated fatty alcohols or fatty acids), polyglycerol derivatives or sugar esters.

Preferred viscosity regulators are NaCl, low molecular weight nonionic surfactants, such as cocamide DEA/MEA and laureth-3, or polymeric high molecular weight associative highly ethoxylated fatty derivatives, such as PEG-200 hydrogenated glyceryl palmitate.

Preferred conditioners are organic quaternary compounds, such as cetrimonium chloride, dicetyldimonium chloride, behentrimonium chloride, distearyldimonium chloride, behentrimonium methosulphate, distearylethyldimonium chloride, palmitamidopropyltrimonium chloride, guar hydroxypropyltrimonium chloride, hydroxypropyl guar hydroxypropyltrimonium chloride or quaternium-80, or also amine derivatives, such as, e.g., aminopropyl dimethicone or stearamidopropyl dimethylamines. A formulation according to the invention can be used alone or in combination with a further or several active substances in cleaning or caring cosmetic formulations for regulating and improving the moisture content of the skin.

Formulations according to the invention can accordingly find use as a skin care, face care, head care, body care, personal care, foot care, hair care, nail care, dental care or oral care product.

Formulations according to the invention can find use in the form of an emulsion, a suspension, a solution, a cream, an ointment, a paste, a gel, an oil, a powder, an aerosol, a pencil, a spray, a cleaning product, a make-up or antisun preparation or a face lotion.

Formulations corresponding to the present invention have a moisturizing and skin-soothing effect. A subject-matter of the invention is accordingly the use of the formulation according to the invention in increasing and/or stabilizing the moisture content of the skin.

Formulations according to the invention decrease the roughness of overtaxed skin. Accordingly, a further subject-matter of the invention is the use of the formulations according to the invention for reducing skin roughness.

The compounds according to formula I can, e.g., be prepared with the process described below.

This process is characterized in that, in a first process stage A, a carboxylic acid according to formula II

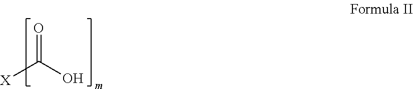

is reacted with an amine of the formula III

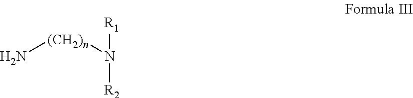

to give an amidoamine according to formula IV:

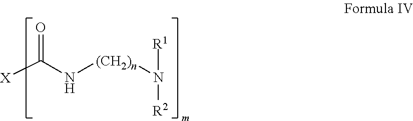

in which n=1 to 6 and m=1 to 4, and R¹ and R² are, independently of one another, identical or different aliphatic hydrocarbon radicals having 1 to 6 carbon atoms and X is an m-valent radical or a covalent bond, with, for m=1, X=H, ethyl, hydroxymethyl, 1-hydroxyethyl, 2-hydroxyethyl, 1-hydroxypropyl, 2-hydroxypropyl or 3-hydroxypropyl, and also, for m=2, X=direct bond, —CH₂—, —CH(OH)—, —CH₂CH(OH)— or —CH(OH)CH(OH)— and X, for m=2, is a direct bond, or a divalent C₁-C₅-hydrocarbon radical which is unsubstituted or substituted with at least one OH group and X, for m_{>}2, is an m-valent C₁-C₅-hydrocarbon radical which is unsubstituted or substituted with at least one OH group, and,

subsequently, in process stage B, the amidoamine of the formula IV obtained in A is reacted with an ω-halocarboxylic acid or its salt, preferably metal salt, especially sodium salt, exhibiting an acid radical according to formula V

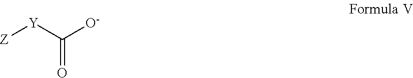

to give the compound of the formula I, in which Z is a halogen and Y is a divalent hydrocarbon radical.

All mono-, di- or polycarboxylic acids or also mixtures of these which meet the requirements mentioned for formula II can be used as carboxylic acids in process stage A. Use may be made, for the preparation of a dizwitterionic compound of the formula I with m=2, as carboxylic acids in process stage A, preferably of oxalic acid (HOOC—COOH), hydroxymalonic acid (HOOC—CH(OH)—COOH), malic acid (HOOC—CH(OH)—CH₂—COOH) and tartaric acid (HOOC—CH(OH)—CH(OH)—COOH), particularly preferably malonic acid (HOOC—CH₂—COOH). Preferred carboxylic acids in process stage A for the preparation of the substance according to formula I with preferred m=1 are lactic acid, propionic acid and glycolic acid; formic acid (HCOOH) is particularly preferred. All suitable amine compounds which meet the requirements of the formula III may be used as amine component. Use is preferably made of 3-(diethylamino)propylamine, 2-(diethylamino)ethylamine or 2-(dimethylamino)ethylamine. Dimethylaminopropylamine (DMAPA) is particularly preferred as amine component.

Preferably, in stage A of the process, an acid component according to formula II is reacted with an amine component according to formula III at a temperature of 90° C. to 220° C., particularly preferably at a temperature of approximately 180° C., to give an amidoamine according to formula IV. Process stage A of the process is particularly preferably carried out using a suitable catalyst. Use is preferably made, as suitable catalysts, of strong base catalysts, such as, e.g., alkoxides; use is particularly preferably made of sodium ethoxide, potassium ethoxide, sodium methoxide and potassium methoxide.

The water formed in the reaction can be removed from the product. The water is preferably distilled off under the reaction conditions and thus removed from the product mixture. At temperatures of below approximately 130° C., in particular, the application of a negative pressure is advantageous in order to accelerate the removal of the water by distillation.

The following reaction scheme shows a possible reaction course of the process stage A.

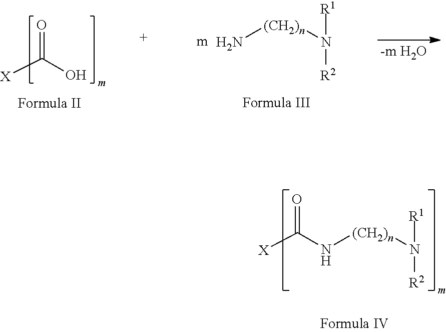

Since the salt mixtures produced in process stage A at the start of the reaction are solid, the acid component according to formula II in the process is preferably added to the introduced amine component according to formula III in the opposite order in comparison with the state of the art.

In the preparation of short-chain amidoamines according to formula IV, the greatly increased exothermicity, in comparison to amidoamines of relatively long-chain fatty acids known from the state of the art, in the formation of the salt between amine according to formula III and acid according to formula II, which is conditioned by the low molecular weights and thereby relatively high molar concentrations, should be borne in mind. With regard to this, especially appropriate process parameters can be applied in process stage A in the form that the addition of the carboxylic acid component to the amine component takes place so slowly that the temperature of the reaction mixture during the addition does not exceed 130° C., preferably 100° C. At higher temperatures, relatively large amounts of the amine component could be stripped out by the water being produced, which can have a negative effect on the stoichiometry of the components used. Preferably, for meeting the temperature ranges mentioned, countercurrent cooling is carried out in order to achieve an economically meaningful metering rate.

The process stage B can be carried out in the presence of a suitable solvent in an amount which guarantees that the reaction mixture can be stirred and pumped at any point in the process. Preferably, the reaction is carried out in the presence of water as solvent. The process stage B is preferably carried out at a temperature of approximately 70-100° C. The halide Z obtained as byproduct can be removed from the reaction solution or remain therein. Should the halide be removed, use may be made, e.g., of precipitation with a suitable solvent or dialysis. The preferred solvent for precipitation is ethanol.

In a preferred embodiment of the process, the halide Z remains in the solution.

Use may be made, as monohalocarboxylic acid or monohalocarboxylic acid salt with an acid radical according to formula V, of all halocarboxylic acids having an acid radical which meets the requirements mentioned for formula V. Particular preference is given, as monohalocarboxylic acid salt according to formula V, to the monochloroacetate.

As already in process stage A, the greatly increased exothermicity conditioned by the low molar mass of the short-chain amidoamine component, in contrast to the process of the state of the art, should also be borne in mind in process stage B. Accordingly, the reaction takes place in process stage B in the form that, during and after complete addition of the halocarboxylic acid component to the amidoamine component, the reaction temperature is maintained at a maximum of approximately 70° C. until the heat of reaction abates, it being possible for countercurrent cooling to be carried out, if appropriate. The following reaction is preferably carried out slightly below the boiling point of the solvent, temperatures in the range of 95-99° C. preferably being used when water is used as solvent.

The following reaction scheme shows a possible reaction course of the process stage B.

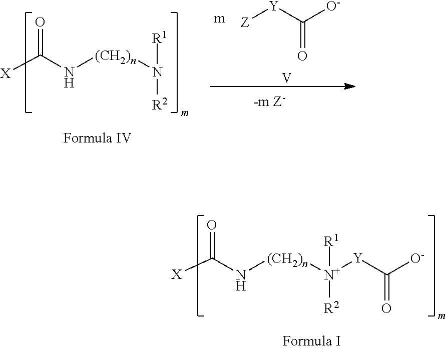

The reaction of amidoamines according to formula IV to give the corresponding compounds according to formula I takes place as described preferably in a solvent. The amidoamines are preferably used in concentrations of 3 to 75%, preferably 5 to 50%. The solution of compounds according to formula I obtained in this process stage can be used with or without further concentration or salt removal stages, e.g. in the manufacture of cosmetic preparations.

In the examples cited subsequently, the present invention is described by way of example without the invention, the range of applications thereof resulting from the complete description and the claims being limited to the embodiments mentioned in the examples.

The following figures of the present application are a constituent of the examples.

### EXAMPLES

### Example 1.1

### Preparation of the Compound 1.1

100 g of formic acid are placed in a 500 ml stirred vessel with a reflux condenser and a nitrogen inlet and rendered inert with nitrogen for approximately 10 minutes. 225 ml of 3-dimethylaminopropylamine are then added with stirring and continuous inerting with nitrogen. Salt formation is exothermic and the mixture heats up to approximately 175° C. and is maintained at this temperature for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 10.4%.

### Example 1.2

### Preparation of the Compound 1.2

133 g of lactic acid are placed in a 500 ml stirred vessel with a reflux condenser and a nitrogen inlet and rendered inert with nitrogen for approximately 10 minutes. 188 ml of 3-dimethylaminopropylamine are then added with stirring and continuous inerting with nitrogen. Salt formation is exothermic and the mixture heats up to approximately 150° C. and is maintained at a temperature of 175° C. for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 8.14%.

### Example 1.3

### Preparation of the Compound 1.3

60 g of acetic acid are placed in a 250 ml stirred vessel with a reflux condenser and a nitrogen inlet and rendered inert with nitrogen for approximately 10 minutes. 120 ml of 3-dimethylaminopropylamine are then added with stirring and continuous inerting with nitrogen. Salt formation is exothermic and the mixture heats up to approximately 150° C. and is maintained at a temperature of 175° C. for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 9.7%.

### Example 1.4

### Preparation of the Compound 1.4

148 g of propionic acid are placed in a 500 ml stirred vessel with a reflux condenser and a nitrogen inlet and rendered inert with nitrogen for approximately 10 minutes. 280 ml of 3-dimethylaminopropylamine are then added with stirring and continuous inerting with nitrogen. Salt formation is exothermic and the mixture heats up to approximately 150° C. and is maintained at a temperature of 175° C. for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 8.91%.

### Example 1.5

### Preparation of the Compound 1.5

110 g of glutaric acid are placed in a 500 ml stirred vessel with a reflux condenser and a nitrogen inlet. 200 ml of 3-dimethylaminopropylamine are then added with stirring and continuous inerting with nitrogen. After the melting of the solid produced, salt formation is exothermic and the mixture heats up to approximately 150° C. and is maintained at a temperature of 175° C. under nitrogen for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 9.47%.

### Example 1.6

### Preparation of the Compound 1.6

90 g of oxalic acid are placed in a 500 ml stirred vessel with a reflux condenser and a nitrogen inlet. 328 ml of 3-dimethylaminopropylamine are then added with stirring and continuous inerting with nitrogen. After the melting of the solid produced, salt formation is exothermic and the mixture heats up to approximately 150° C. and is maintained at a temperature of 175° C. under nitrogen for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 12.5%.

### Example 1.7

### Preparation of the Compound 1.7

104 g of malonic acid are placed in a 500 ml stirred vessel with a reflux condenser and a nitrogen inlet. 280 ml of 3-dimethylaminopropylamine are then added with stirring and continuous inerting with nitrogen. After the melting of the solid produced, salt formation is exothermic and the mixture heats up to approximately 140° C. and is maintained at a temperature of 175° C. under nitrogen for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 9.84%.

### Example 1.8

### Preparation of the Compound 1.8

76 g of glycolic acid are placed in a 500 ml stirred vessel with a reflux condenser and a nitrogen inlet. 135 ml of 3-dimethylaminopropylamine are then added with stirring and continuous inerting with nitrogen. After the melting of the solid produced, salt formation is exothermic and the mixture heats up to approximately 140° C. and is maintained at a temperature of 175° C. under nitrogen for approximately 4-5 h. During this time, water produced in the reaction is removed from the mixture via a column. When, from the acid number, a degree of conversion of approximately 98% is achieved, excess DMAPA is removed by means of vacuum distillation. The content of tertiary nitrogen in the purified final product is 8.96%.

### Example 2.1

### Preparation of the Compound 2.1

91 g of sodium monochloroacetate and 191 g of water are weighed out in a 500 ml four-necked round-bottomed flask equipped with a stirrer, thermometer, reflux condenser and dropping funnel and heated to 40° C. 100 g of the amidoamine from Example 1.1 are added and the reaction mixture is maintained at a temperature of 70° C. until the heat of reaction abates. The reaction mixture is then heated to 98° C. After approximately 7 h, the content of residual amidoamine is less than 0.5%. 382 g of an aqueous solution with the following composition are obtained:

Compound 2.1: 38.1%

NaCl: 11.9%

Water: 50%

Appearance: liquid, clear

### Example 2.2

### Preparation of the Compound 2.2

70 g of sodium monochloroacetate and 170 g of water are weighed out in a 500 ml four-necked round-bottomed flask equipped with a stirrer, thermometer, reflux condenser and dropping funnel and heated to 40° C. 100 g of the amidoamine from Example 1.2 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

340 g of an aqueous solution with the following composition are obtained:

Compound 2.2: 39.7%

NaCl: 10.3%

Water: 50%

Appearance: liquid, clear

### Example 2.3

### Preparation of the Compound 2.3

83 g of sodium monochloroacetate and 183 g of water are weighed out in a 500 ml four-necked round-bottomed flask equipped with a stirrer, thermometer, reflux condenser and dropping funnel and heated to 40° C. 100 g of the amidoamine from Example 1.3 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

364 g of an aqueous solution with the following composition are obtained:

Compound 2.3: 38.5%

NaCl: 11.5%

Water: 50%

Appearance: liquid, clear

### Example 2.4

### Preparation of the Compound 2.4

153 g of sodium monochloroacetate and 353 g of water are weighed out in a 1000 ml four-necked round-bottomed flask equipped with a stirrer, thermometer, reflux condenser and dropping funnel and heated to 40° C. 200 g of the amidoamine from Example 1.4 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

706 g of an aqueous solution with the following composition are obtained:

Compound 2.4: 39.2%

NaCl: 10.8%

Water: 50%

Appearance: liquid, clear

### Example 2.5

### Preparation of the Compound 2.5

82 g of sodium monochloroacetate and 182 g of water are weighed out in a 500 ml four-necked round-bottomed flask equipped with a stirrer, thermometer, reflux condenser and dropping funnel and heated to 40° C. 100 g of the amidoamine from Example 1.5 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

364 g of an aqueous solution with the following composition are obtained:

Compound 2.5: 38.7%

NaCl: 11.3%

Water: 50%

Appearance: liquid, clear

### Example 2.6

### Preparation of the Compound 2.6

107 g of sodium monochloroacetate and 207 g of water are weighed out in a 500 ml four-necked round-bottomed flask equipped with a stirrer, thermometer, reflux condenser and dropping funnel and heated to 40° C. 100 g of the amidoamine from Example 1.6 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

414 g of an aqueous solution with the following composition are obtained:

Compound 2.6: 35.9%

NaCl: 14.1%

Water: 50%

Appearance: liquid, clear

### Example 2.7

### Preparation of the Compound 2.7

85 g of sodium monochloroacetate and 185 g of water are weighed out in a 500 ml four-necked round-bottomed flask equipped with a stirrer, thermometer, reflux condenser and dropping funnel and heated to 40° C. 100 g of the amidoamine from Example 1.7 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

370 g of an aqueous solution with the following composition are obtained:

Compound 2.7: 38.5%

NaCl: 11.5%

Water: 50%

Appearance: liquid, clear

### Example 2.8

### Preparation of the Compound 2.8

115 g of sodium monochloroacetate and 265 g of water are weighed out in a 1000 ml four-necked round-bottomed flask equipped with a stirrer, thermometer, reflux condenser and dropping funnel and heated to 40° C. 150 g of the amidoamine from Example 1.8 are added. The reaction mixture is then heated to 70° C. and the temperature is maintained until the heat of reaction abates. The reaction mixture is subsequently heated to 98° C. After approximately 7 h, the content of residual amidoamine is less than 0.5%.

530 g of an aqueous solution with the following composition are obtained:

Compound 2.8: 38.5%

NaCl: 11.5%

Water: 50%

Appearance: liquid, clear

Formulation examples for caring formulations:

Proof of effectiveness of the short-chain zwitterionic compounds in the more detailed discussion of the invention:

In order to be able to characterize the skin care properties of the compounds 2.1 to 2.8, various in vitro tests were carried out on skin models (reconstructed human epidermis, company: SkinEthic).

### Example 3.1

### Lactate Dehydrogenase Release (LDH Release)

The occurrence of LDH in the cell culture medium is a sure sign of damage to the cytoplasmic membrane of the cells and accordingly of damage to the epidermal cell layer. Furthermore, it is known that an escape of this enzyme represents the “point of no return” for the cell and thus indicates that the damage is irreversible.

The LDH concentration was determined with a commercially available test kit (LDH test kit, Roche Diagnostics, Mannheim, Germany).

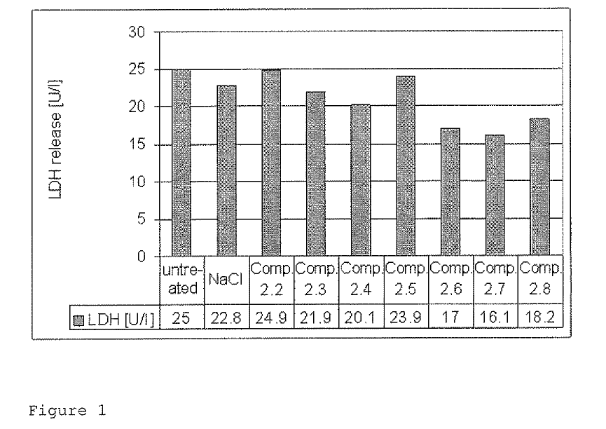

The test formulation was applied twice to the skin model with an interval of 24 h. FIG. 1 represents the LDH release 24 h after the final application.

Test formulation 3.1: A 4% aqueous solution of the short-chain zwitterionic compounds was applied. Since the compounds comprise approximately 0.3% of sodium chloride per 1% of active substance, the corresponding sodium chloride concentration was also tested.

The LDH release from the cells was unchanged by the application of the compounds or even, in comparison with the untreated skin model, slightly lower. This means that the short-chain zwitterionic compounds do not attack the cell membrane and thus do not cause cell damage.

### Example 3.2

### LDH Release after Damaging the Cells with SDS

Sodium dodecyl sulphate (SDS) is known for attacking the cell membrane and for resulting in increased LDH release. The experiment described below should investigate to what extent compound 2.1 can protect the cells after damaging with SDS.

The skin model was damaged with SDS for 40 min. Subsequently, the test formulation, an O/W cream with 1 or 4% of compound 2.1, was applied. The LDH release was measured 24 h and 48 h after application of the test formulation.

Test formulation 3.2:

| | |
|---|---|
| Polyglyceryl-3 methylglucose distearate | 3.0% |
| Glyceryl stearate | 2.0% |
| Stearyl alcohol | 1.0% |
| Cetearyl ethylhexanoate | 5.0% |
| Mineral oil | 14.0% |
| Compound 2.1 | 1.0/4.0% |
| Water | ad 100.0% |

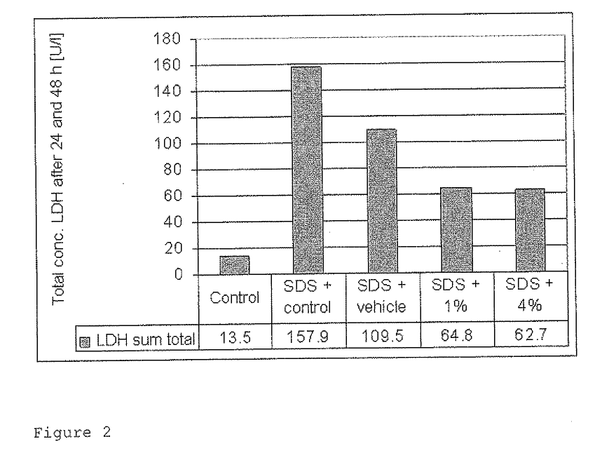

FIG. 2 represents the total concentration of LDH after 24 and 48 h.

As a result of damage by SDS, the LDH release greatly increases, as expected. This increase was clearly reduced if the test formulation was applied directly after damaging. A positive effect was already recognizable with the vehicle but clearly intensified again if the formulation comprised compound 2.1. In this connection, 1% of the compound according to the invention already appears to be sufficient since no clear increase in the effectiveness was recognizable with 4%.

### Example 3.3

### IL-1α Release after Damaging with SDS

IL-1α is a neurotransmitter which plays a central role in inflammatory reactions in the body. Sodium lauryl sulphate (SDS) is a skin-irritating surfactant which can give rise to irritant contact dermatitis in man, is used as model irritant in proband studies and, inter alia, induces the release of IL-1α. The IL-1α concentration was determined with a commercially available test kit (human IL-1α immunoassay, R&D Systems GmbH, Wiesbaden, Germany).

The test formulation, a 4% aqueous solution of the short-chain zwitterionic compounds, was applied to the skin model. 24 h after application, the skin model was damaged for 40 min with 0.25% SDS solution. Subsequently, the test formulation was applied a second time. After incubating for a further period of 24 h, the cytokine IL-1α released was determined.

Since the test solutions comprise approximately 0.3% of NaCl per 1% of active substance, a correspondingly concentrated sodium chloride solution was also investigated.

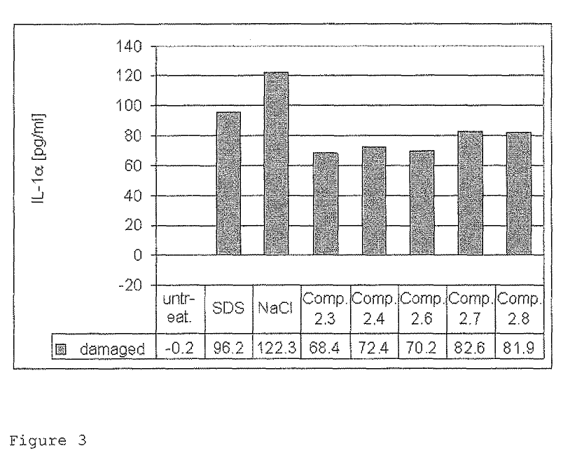

FIG. 3 shows the test results for the IL-1α concentration 24 h after damaging.

All compounds tested reduced the release of the inflammatory marker IL-1α, i.e. it can be assumed therefrom that the short-chain zwitterionic compounds have anti-inflammatory properties.

### Example 3.4

### Anti-inflammatory Action of an O/W Cream with Compound 2.1

It should be investigated whether the anti-inflammatory effect of the short-chain zwitterionic compounds also appears when used from a cosmetic formulation. For this, skin models were damaged with SDS. Subsequently, the test formulation with 1% and 4% of compound 2.1 was applied. The IL-1α concentration was determined 24 and 48 h after application.

Test formulation:

| | |
|---|---|
| Polyglyceryl-3 methylglucose distearate | 3.0% |
| Glyceryl stearate | 2.0% |
| Stearyl alcohol | 1.0% |
| Cetearyl ethylhexanoate | 5.0% |
| Mineral oil | 14.0% |
| Compound 2.1 | 1.0/4.0% |
| Water | ad 100.0% |

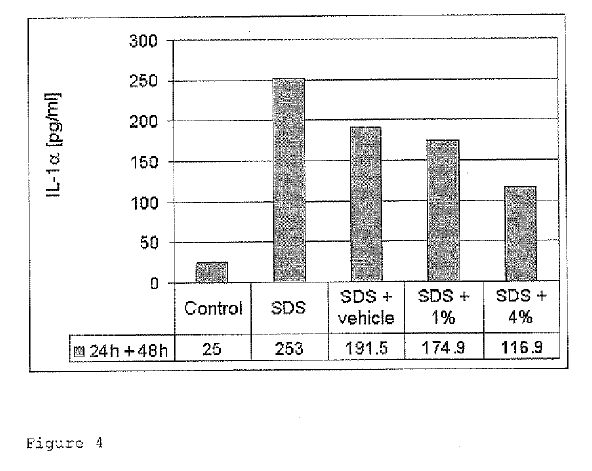

FIG. 4 represents the overall IL-1α concentration after 24 and 48 h.

As expected, the formation of the cytokine IL-1α was greatly increased by damaging with SDS. This increase was more strongly reduced in a concentration-dependent fashion by addition of compound 2.1, so that, on using the zwitterionic compounds from an O/W emulsion, a clearly anti-inflammatory action also appears.

### Example 3.5

### XTT Test

The XTT test is based on the ability of the cells to reduce the dye XTT, which can be detected photometrically. This reaction is catalyzed by mitochondrial succinate dehydrogenase and requires NAD(P)H, which can only be formed by metabolically active cells. To sum up, the XTT test describes the viability of the cells.

The XTT test was carried out with a commercially available test kit and took place according to the manufacturer's instructions (XTT Test, Roche Diagnostics, Mannheim, Germany).

The test formulation, a 4% aqueous solution of the short-chain zwitterionic compounds, was applied to the skin model twice with an interval of 24 h. 24 h after the second application, the XTT concentration was determined. In addition to the zwitterionic compounds, the concentration of sodium chloride correspondingly present in the test solutions was again tested. 0.25% SDS was used as negative control.

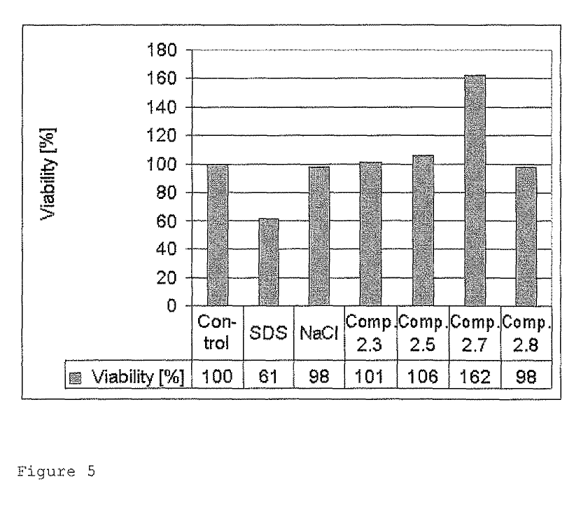

FIG. 5 represents the viability of the cells, based on the control.

It is seen that the viability of the cells was not negatively influenced by the zwitterionic test substances. On the contrary, the viability was even positively influenced by compound 2.7.

### Example 3.6

### XTT Test with an O/W Cream with Compound 2.1

The skin model was first damaged with 0.25% SDS for 40 min. Subsequently, the test formulations were applied. After incubating for a time of 24 h, the viability of the cells was determined using the XTT test.

Test formulation: see Example 3.2.

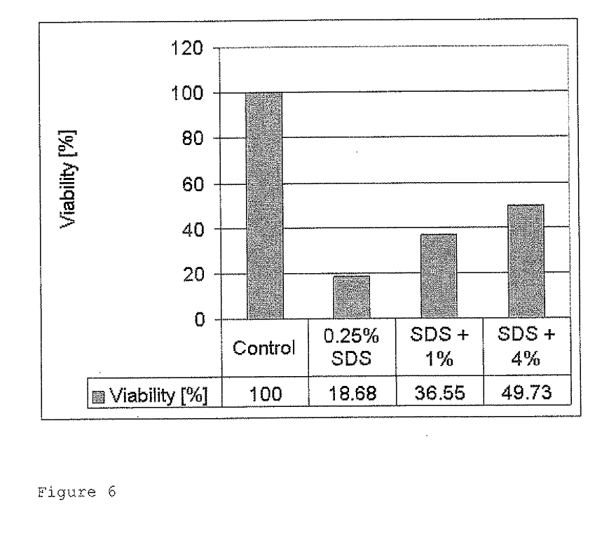

FIG. 6 represents the viability of the test formulations, based on the control, i.e. untreated cells.

It turned out that the viability of the skin cells was greatly reduced by damaging with SDS. The viability could be more than redoubled by the subsequent treatment with compound 2.1.

### Example 3.7

### Water Retention Capacity on an IMS Film

The determination of the water retention capacity of active substances using the IMS film represents a simple screening test by which the moisturizing properties of active substances can be very satisfactorily investigated. The measurement is based on the following principle: The IMS film is a membrane which is covered with peptides, lipids and polymers and represents a greatly simplified skin model. The active substance interacts out of the formulation with the film. Combining with water takes place and thus the evaporation of the water is prevented or made more difficult.

Test formulation 3.7:

| | |
|---|---|
| Ceteareth-25 | 2.0% |
| Glyceryl stearate | 4.0% |
| Cetearyl alcohol | 2.0% |
| Ethylhexyl stearate | 8.5% |
| Caprylic/capric triglycerides | 8.5% |
| Short-chain zwitterionic compounds | 5.0% |
| Water | ad 100.0% |

Implementation:
1. The weight of the IMS membrane is determined (W1).
2. The test formulation is applied, the film is again weighed (W2) and the film is subsequently incubated at 21-22° C. and 72% R.H. for 4 hours.
3. After the 4 hours have passed, the film is again weighed (W3).
4. The water retention capacity of the formulation (WR) is calculated as follows:

WR=(W3−W1)/(W2−W1)*100−100

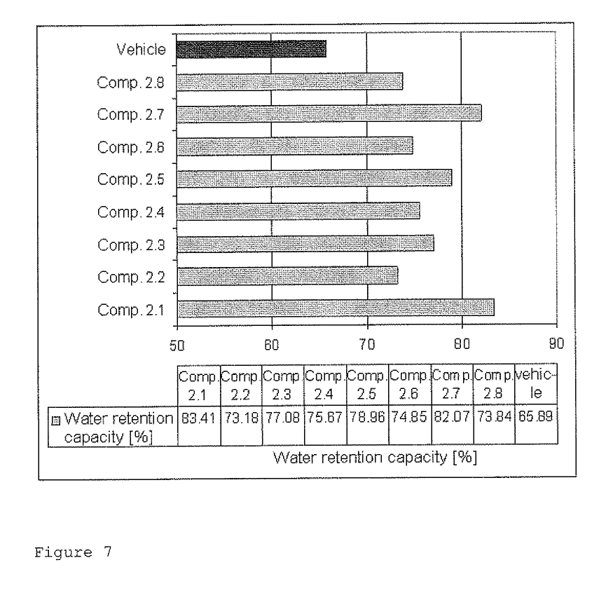

FIG. 7 represents the data measured for the water retention capacity of different short-chain zwitterionic compounds.

All zwitterionic compounds investigated significantly improve the water retention capacity in comparison with the vehicle. This property is particularly strongly pronounced with the compounds 2.1, 2.5 and 2.7. However, even the other short-chain zwitterionic compounds showed a very good water retention capacity.

Example 3.8 In vivo moisturizer properties of short-chain zwitterionic compounds:

Since the very good results obtained with compound 2.1 in the determination of the water retention capacity on an IMS film indicate very good moisturizing properties, the moisturizer properties were also determined in vivo in the next step.

The skin moisture is determined in normal fashion using a corneometer.

With the corneometer principle, the skin moisture of the “outer layer” of the epidermis (stratum corneum) is determined by means of a capacity measurement. This principle is based on the fact that the dielectric constants of water and other substances differ. An appropriately shaped measuring capacitor reacts with different changes in capacity on the samples introduced into its sensing volume, which changes in capacity are recorded and evaluated fully automatically by the device. The active probe coated with special glass is pressed against the place on the skin to be measured and, after 1 second, the value measured by the corneometer, thus the degree of moisture on the skin surface, appears on the display

(www.dermatest.de/de/ueberuns.html).

For the experiments described here, use is made of a corneometer CM 825 from Courage & Khazaka. The skin moisture was measured before and 2 hours after application of the test formulations. For this, 4 test panels were each time highlighted on the forearms of 14 probands, on which the different test formulations were applied. Before each measurement, the probands had to spend at least 15 min in a climate-controlled chamber (21-22° C., 55% R.H.).

The difference in the corneometer values before and after application of the test formulations was calculated. The higher this value, the better the moisturizing properties of the active substance.

Test formulation 3.8:

| | | |
|---|---|---|
| Ceteareth-25 | 2.0% | 2.0% |
| Glyceryl stearate | 4.0% | 4.0% |
| Cetearyl alcohol | 2.0% | 2.0% |
| Ethylhexyl stearate | 8.5% | 8.5% |
| Caprylic/capric triglycerides | 8.5% | 8.5% |
| Compound 2.1 | 1.0/4.0% | — |
| Water | ad 100.0% | ad 100.0% |

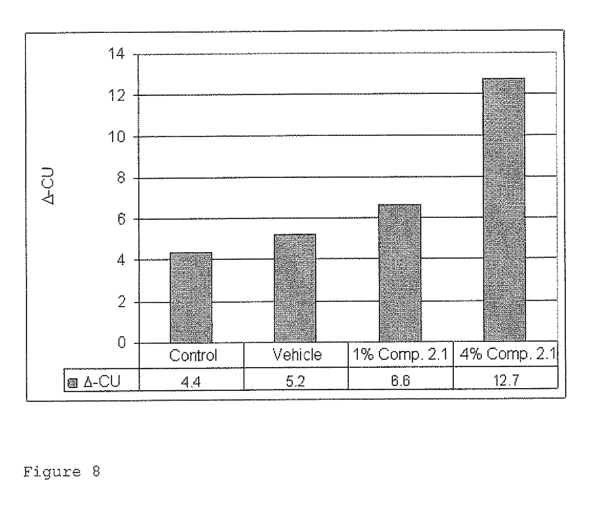

FIG. 8 represents the increase in the corneometer units (ΔCU) 2 hours after application of the test formulations.

Compound 2.1 very clearly increased the skin moisture. The effectiveness clearly increased with increasing concentration of use. It could thus be shown that compound 2.1 has very good moisturizing properties.

### Example 3.9

### Long-Term Moisturizing Effect of Compound 2.1

In order to investigate the long-term effect of the moisturizing properties of compound 2.1, a study lasting two weeks was carried out with 12 probands. The probands received 2 formulations, one with 2% of compound 2.1 and one without active substance. They had to apply each of these formulations on the inner side of a forearm twice daily. The moisture content was measured before beginning to use and also after 2 weeks.

The moisture content was determined using a corneometer CM 825 (Courage & Khazaka). Before each measurement, the probands had to stay in a climate-controlled chamber (21-22° C., 55% R.H.) for at least 15 min. The difference in the corneometer units with respect to the starting value was calculated each time (ΔCU).

Test formulation 3.9:

| | |
|---|---|
| Ceteareth-25 | 2.0% |
| Glyceryl stearate | 4.0% |
| Cetearyl alcohol | 2.0% |
| Ethylhexyl stearate | 8.5% |
| Caprylic/capric triglycerides | 8.5% |
| Compound 2.1 | 0/2.0% |
| Water | ad 100.0% |

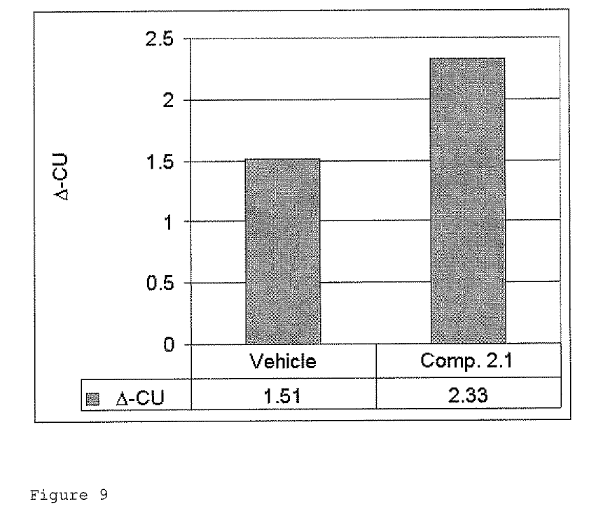

FIG. 9 represents the ΔCU values after application of the test formulations for 2 weeks.

The moisture content is already somewhat improved by the vehicle. This effect is further enhanced by compound 2.1.

### Example 3.10

### Influence of Compound 2.1 on the Skin Roughness

The skin roughness can be quantified in a simple way by means of tape stripping. The rougher the skin surface, e.g. because the skin lipid barrier is damaged, the weaker the binding of the skin cells. In some cases, with very rough skin, this can be seen with the naked eye. The outermost corneocytes are removed by tape stripping. In this connection, the more corneocytes stuck to the tape, the rougher the skin. The corneocytes are subsequently determined quantitatively using a commercially available Bradford test. This is based on the following principle: the triphenylmethane dye Coomassie Brilliant Blue G-250 (CBBG) forms complexes in acid solution both with the cationic and the nonpolar hydrophobic side chains of the proteins. The absorption spectrum of the nonbonded (cationic) red-coloured form has an absorption maximum at 470 nm. By complex formation with proteins, the dye is stabilized in its blue nonprotonated anionic sulphate form and the absorption spectrum shifts to an absorption maximum at 595 nm. Since the extinction coefficient of the dye/protein complex is in addition very much higher than that of the free dye, the increase in the absorption at 595 nm through the formation of the complex can be measured photometrically with high sensitivity against the free dye reagent and is a measure of the protein concentration of the solution.

The study was carried out with 12 probands who received two test formulations, one with 2% compound 2.1 and one without. They had to apply each of these formulations twice daily on the inner side of a forearm. Tape strips were taken and analyzed before the beginning of the application and also after 2 weeks and after 4 weeks.

Test formulation: see Example 3.9

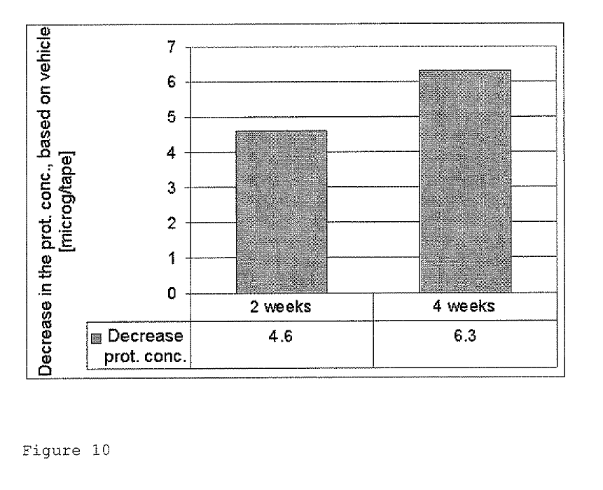

FIG. 10 represents the reduction in the amount of protein, based on the vehicle.

A clear reduction in the skin roughness by the formulation with compound 2.1 can be already recognized after using for two weeks, compared with the vehicle. This effect becomes even more pronounced during the further use.

Formulation examples for cleaning formulations:

### Example 4.1

### Panel Test

Test formulation body cleansing agent:

| Formulation | 4.1a | 4.1b |
|---|---|---|
| Sodium lauryl ether sulphate | 6% | 6% |
| Cocamidopropyl betaine | 6% | 6% |
| Compound 2.1 | 0.5% | |
| Water | ad 100% | ad 100% |
| Phenonip | 0.2% | 0.2% |

In order to determine the influence of the formulations 4.1a and 4.1b on the moisture content of the skin, the formulations 4.1a and 4.1b were investigated in a forearm washing test.

The test panel consisted of 15 test subjects.

The test subjects were instructed not to use any cosmetic products (shower bath, body lotion) on the forearms from 3 days before the beginning of the test.

The test was carried out over 5 days (Mo-Fr).

The starting measurement was taken on the afternoon of the first day 4 hours after defined prewashing with formulation 3.

Three washing operations were carried out per day under defined conditions.

A control measurement was taken on the second day after 3 washing operations.

The concluding measurement was taken on the 5^{th} day 4 hours after the 11^{th} washing operation.

Before the respective measurements, the test subjects spent at least 20 min in a climate-controlled chamber.

The corneometer methods of measurement were carried out according to the procedure described above.

Test results:

| | Δ Corneometry value |
|---|---|
| Formulation 4.1a | −1.0 |
| Formulation 4.1b | −5.1 |
| Untreated | −0.9 |

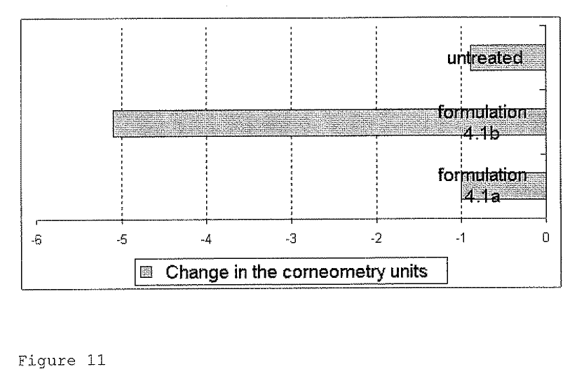

FIG. 11 represents the values listed above. It is obvious, from FIG. 11, that the decrease in the corneometry values observed during a skin cleaning application returns, during the use of formulation 4.1a, to a value observed for untreated skin. Without the use of compound 2.1, a typical significant fall in skin moisture is observed.

1. A formulation comprising at least one compound according to formula I 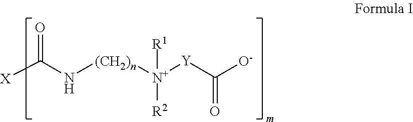
in which
n=1 to 6,
m=1 to 4,
R¹ and R² are, independently of one another, identical or different aliphatic hydrocarbon radicals having 1 to 6 carbon atoms,
Y is a divalent hydrocarbon radical, and
X is an m-valent radical or a covalent bond, with
X, for m=1, being a hydrogen or a C₁-C₄-hydrocarbon which is unsubstituted or substituted with at least one OH group and
X, for m=2, being a direct covalent bond or a divalent C₁-C₅-hydrocarbon radical which is unsubstituted or substituted with at least one OH group, and
X, for m>2, being an m-valent C₁-C₅-hydrocarbon radical which is unsubstituted or substituted with at least one OH group,
and/or a stereoisomeric form of the compound according to formula I.

2. The formulation according to claim 1, wherein m=2 and X=CH₂.

3. The formulation according to claim 1, wherein m=1 and X=H.

4. The formulation according to claim 1, wherein n=3, m 2, R¹=R²═CH₃, Y═CH₂ and X═CH₂.

5. The formulation according to claim 1, wherein n=3, m=1, R¹=R²═CH₃, Y═CH₂ and X═H.

6. The formulation according to claim 1, wherein the at least one compound of formula I is present in an amount of 0.05 to 10% by weight based on the total formulation.

7. The formulation according to claim 1, further comprising at least one additional component selected from the group consisting of
emollients
emulsifiers and surfactants
thickeners/viscosity regulators/stabilizers
UV/light screening agents
antioxidants
hydrotropes (or polyols)
solids
pearlescent additives
deodorant and antiperspirant active substances
insect repellents
self-tanning agents
preservatives
conditioners
fragrances
colorants
biogenic active substances
care additives, and
solvents.

8. A cosmetic including at least the formulation according to claim 1.

9. The formulation according to claim 1, wherein said formulation is a component of a skin care, face care, head care, body care, personal care, foot care, hair care, nail care, dental care or oral care product.

10. The formulation according to claim 1, wherein said formulation is in the form of an emulsion, a suspension, a solution, a cream, an ointment, a paste, a gel, an oil, a powder, an aerosol, a pencil, a spray, a cleaning product, a make-up or antisun preparation or a face lotion.

11. A method for increasing and/or stabilizing moisture content of the skin comprising applying the formulation according to claim 1 to skin wherein said skin after application of said formulation has a moisture content that is equal to or greater than the moisture content of the skin prior to said application.

12. A method for reducing skin roughness comprising applying the formulation according to claim 1 to skin wherein said skin after application of said formulation has a roughness that is less than the roughness of the skin prior to said application.
